SEQUENCE LISTING (6 sequences)

SEQ ID NO: 1 - protein, 6 aa, Artificial Sequence
  fragment of human ghrelin peptide
GSSFLS

SEQ ID NO: 2 - protein, 6 aa, Artificial Sequence
  labelled fragment of human ghrelin peptide
GSSFLS

SEQ ID NO: 3 - protein, 6 aa, Artificial Sequence
  labelled mimic of human ghrelin peptide
GCSFLS

SEQ ID NO: 4 - protein, 6 aa, Artificial Sequence
  labelled mimic of human ghrelin peptide
GSSFCS

SEQ ID NO: 5 - protein, 6 aa, Artificial Sequence
  labelled mimic of human ghrelin peptide
GSSFLC

SEQ ID NO: 6 - protein, 5 aa, Artificial Sequence
  acylated fragment of human ghrelin peptide
SSFLC

(19) United States
(12) Patent Application Publication
(10) Pub. No.: US 2017/0056373 A1
(43) Pub. Date: Mar. 2, 2017

(54) GHRELIN O-ACYLTRANSFERASE INHIBITORS

(71) Applicant: James Hougland, Fayetteville, NY (US)

(72) Inventor: James Hougland, Fayetteville, NY (US)

(73) Assignee: SYRACUSE UNIVERSITY, SYRACUSE, NY (US)

(21) Appl. No.: 15/254,267

(22) Filed: Sep. 1, 2016

Related U.S. Application Data
(60) Provisional application No. 62/212,647, filed on Sep. 1, 2015.

(51) Int. Cl. A61K 31/4164; A61K 31/277

(52) U.S. Cl. CPC A61K 31/4164; A61K 31/277

(57) ABSTRACT

Small molecule ghrelin O-acyltransferase inhibitors found using an assay to detect ghrelin O-acyltransferase activity using an acrylodan-labeled peptide mimic of ghrelin that provides for high-throughput screening for ghrelin O-acyltransferase inhibitors and detection via high performance liquid chromatography. The newly discovered class of synthetic triterpenoids efficiently inhibits ghrelin acylation by GOAT and function as covalent reversible inhibitors of GOAT. In cell studies, the most potent members of this family of compounds efficiently block ghrelin acylation at submicromolar concentrations and offer a foundation for continued development and evaluation of novel hGOAT inhibitors as therapeutics targeting disorders such obesity, type II diabetes, gastroparesis, and Prader-Willi syndrome.

### CROSS-REFERENCE TO RELATED APPLICATIONS

The present application claims priority to U.S. Provisional No. 62/212,647, filed on Sep. 1, 2015.

### BACKGROUND OF THE INVENTION

1. Field of the Invention

The present invention relates to ghrelin O-acyltransferase inhibitors and, more particularly, to small molecule inhibitors of ghrelin O-acyltransferase.

2. Description of the Related Art

The ghrelin—ghrelin O-acyltransferase (GOAT) system has been implicated as a potential target for pharmacologic modulation of a number of disorders, including obesity and diabetes. Ghrelin is a peptide hormone involved in appetite regulation, glucose metabolism, and also potentially learning and memory. To transduce ghrelin-dependent signaling, ghrelin requires octanoylation of its serine 3 residue (GSSFLS . . . ) (SEQ. ID No. 1) to bind and activate its cognate receptor. This acylation is catalyzed by ghrelin O-acyltransferase (GOAT), a member of the MBOAT family of integral membrane enzymes.

With ghrelin impacting multiple physiological pathways, the ghrelin-GOAT system presents an attractive therapeutic target. For example, ghrelin-linked signaling may be involved in various diseases, such as obesity, appetite dysregulation, type II diabetes, and other conditions, as well as involved in learning and memory, depression, and Parkinson's disease. However, the lack of information regarding GOAT structure and catalytic mechanism renders GOAT inhibitor design and optimization difficult.

### BRIEF SUMMARY OF THE INVENTION

The present invention comprises a series of a ghrelin O-acyltransferase inhibitors found using an assay for human ghrelin O-acyltransferase activity. The first step of an assay according to the present invention is to design a synthetic peptide substrate that mimics the N-terminal sequence of ghrelin. The next step in the present invention is to attach an environmentally-sensitive fluorophore to the C-terminal amino acid of the substrate through chemoselective ligation. Then, upon octanoylation of serine 3, the peptide substrate becomes more hydrophobic, leading to an increase in the fluorescence of the environmentally-sensitive fluorophore. Alternatively, the presence of any acylation of the fluorescent peptide substrate can be detected via reverse-phase high performance liquid chromatography (HPLC).

Using this assay, several small molecule ghrelin O-acyltransferase inhibitors were found. A screen of a library of small molecules with diverse structures revealed a novel small molecule hGOAT inhibitor, CDDO-Im, with subsequent structure-activity analysis revealing this compound and related molecules function as reversible covalent inhibitors of GOAT. This new class of inhibitors exhibits robust inhibition of ghrelin octanoylation in both enzyme-and cell-based assays, and previous clinical studies employing these compounds establish the suitability of these inhibitors for human treatment. Our study establishes these synthetic triterpenoids as potential “first-in-class” molecules targeting ghrelin signaling and offers the foundation for continued development and evaluation of novel hGOAT inhibitors as therapeutics targeting diabetes and obesity.

### BRIEF DESCRIPTION OF THE SEVERAL VIEWS OF THE DRAWING(S)

The present invention will be more fully understood and appreciated by reading the following Detailed Description in conjunction with the accompanying drawings, in which:

FIG. 1 is a schematic of ghrelin acylation by ghrelin O-acyltransferase.

FIG. 2 is a schematic of a fluorescently labeled ghrelin fragment according to the present invention.

FIGS. 3(a) through 3(c) are a schematics of screening of Diversity IV library compounds to reveal a novel small molecule hGOAT inhibitor. FIG. 3(a) is a fluorescence-based hGOAT activity assay used for compound screening. FIG. 3(b) is a protocol for screening assay to identify hGOAT inhibitors. FIG. 3(c) is a schematic of a structure of CDDO-Im (1), the initial hit from the Diversity IV library and inhibition of hGOAT octanoylation activity by CDDO-Im (1).;

FIG. 4 is chart of synthetic oleanate triterpenoids evaluated for potential inhibitory activity towards GOAT according to the present invention;

FIG. 5 is a schematic showing multiple reported CDDO derivatives with potential inhibitory activity towards GOAT;

FIG. 6 is a schematic showing that multiple CDDO derivatives effectively inhibit hGOAT. Structures and IC₅₀ values for CDDO derivatives with substitutions at the carboxyl group at position 28: R=imidazole (CDDO-Im, 1); R=methyl ester (CDDO-Me, 2); R=ethylamide (CDDO-EA, 3); R=trifluoroethylamide (CDDO-TFEA, 4); R=carboxylic acid (CDDO, 5);

FIGS. 7(a) and 7(b) are graphs showing that ghrelin acylation is inhibited by CDDO-EA and CDDO-Me in a cell-based assay. FIG. 7(a) shows dose-dependent reduction of acyl ghrelin concentrations in GOAT/preproghrelin transfected HEK293FT cells following 24 hours incubation, normalized to the DMSO vehicle: CDDO (compound 5, white bar); CDDO-EA (compound 3, crosshatched bar); and CDDO-Me (compound 2, black bar). Acyl ghrelin concentrations were determined using a commercially available ELISA kit (Millipore). Error bars reflect the standard deviation from three independent measurements. FIG. 7(b) shows cell viability for GOAT/preproghrelin transfected HEK293FT cells cell incubation following 24 hours incubation, normalized to the DMSO vehicle: CDDO (compound 5, white bar); CDDO-EA (compound 3, crosshatched bar); and CDDO-Me (compound 2, black bar). Cell viability was determined by Alamar Blue assay using the manufacturers protocol (Thermo-Fisher). Error bars reflect the standard deviation from three independent measurements;

FIGS. 8(a) through 8(c) are a series of graphs of the inhibition of hGOAT activity by CDDO derivatives. FIG. 8(a) shows inhibition of hGOAT octanoylation activity by CDDO-Me (compound 2). FIG. 8(b) shows inhibition of hGOAT octanoylation activity by CDDO-EA (compound 3). FIG. 8(c) show inhibition of hGOAT octanoylation activity by CDDO-TFEA (compound 4); and

FIGS. 9(a) and 9(b) are a series of graphs of the inhibition of ghrelin octanoylation by CDDO derivatives in a cell-based assay. FIG. 9(a) Inhibition of hGOAT octanoylation activity by CDDO-EA (compound 3). FIG. 9(b) Inhibition of hGOAT octanoylation activity by CDDO-Me (compound 2).

### DETAILED DESCRIPTION OF THE INVENTION

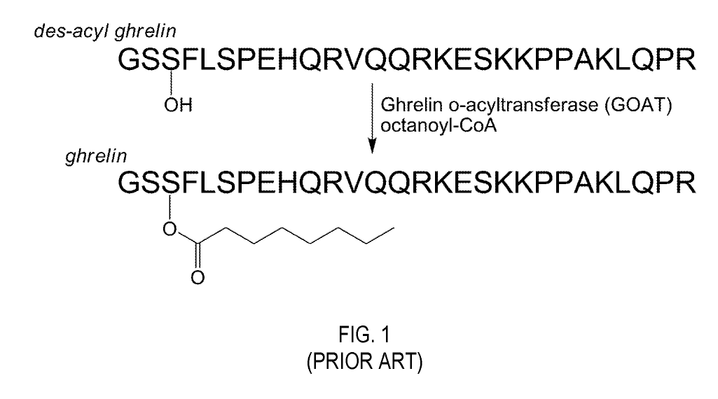

The present comprises certain human ghrelin inhibits identified using a fluorescence-based assay for ghrelin acylation that greatly simplifies mechanistic studies of GOAT and allows for high-throughput screening of potential GOAT inhibitors. As seen in FIG. 1, GOAT catalyzes the n-octanoyl transfer to ghrelin. Only acylated ghrelin binds to its cognate receptor and a large fraction of ghrelin species in circulation is des-acyl ghrelin. GOAT-catalyzed acylation thus potentially serves as a mechanism for controlling ghrelin-mediated signaling.

The first step of an assay according to the present invention is to design one or more synthetic peptide substrates that mimic the N-terminal sequence of ghrelin. The next step in the present invention is to attach an environmentally-sensitive fluorophore to the C-terminal amino acid of the substrate through chemoselective ligation. Then, upon octanoylation of serine 3 of ghrelin by GOAT, the peptide substrate becomes more hydrophobic leading to an increase in the fluorescence of the environmentally-sensitive fluorophore and increased peptide substrate retention time on reverse-phase HPLC.

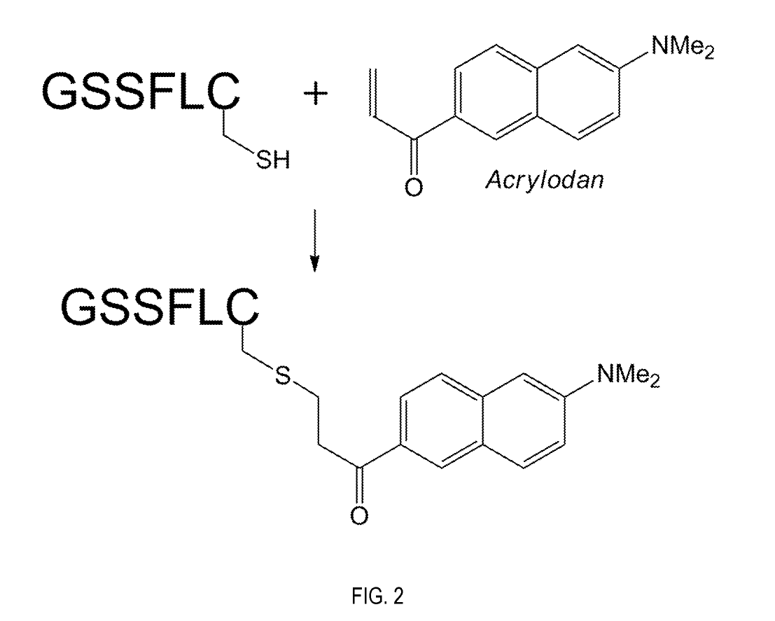

As seen in FIG. 2, an exemplary peptide substrate for evaluating GOAT-catalyzed acylation was created by forming an acrylodan (AcDan) labeled fragment of the ghrelin peptide. A series of ghrelin mimic peptides were created along these lines with covalently attached fluorophores to serve as fluorescent substrates according the present invention, and tested for activity with hGOAT, as detailed in Table 1 below.

TABLE 1
Peptide substrates for fluorescence-based assay
of GOAT activity

| | | Active |
|---|---|---|
| Substrate | Peptide Sequence | with hGOAT? |
| 1-Dansyl | Dansyl-GSSFLS | No |
| | (SEQ. ID No. 2) | |
| AcDan-2 | GC_{AcDan}SFLS | No |
| | (SEQ. ID No. 3) | |
| AcDan-5 | GSSFC_{AcDan}S | Yes |
| | (SEQ. ID No. 4) | |
| AcDan-6 | GSSFLC_{AcDan} | Yes |
| | (SEQ. ID No. 5) | |

It should be recognized by those of skill in the art that other environmentally sensitive fluorophores (including, but not limited to, dansyl, dapoxyl, coumarin fluorophores) may be employed. Alternatively, fluorophore coupling chemistry may be used to develop fluorescent ghrelin analog substrates with higher reactivity and fluorescence yields.

The peptides are derived from the N-terminal sequence of ghrelin (GSSFLS) (SEQ. ID No. 1) and include all amino acids currently proposed to be important for ghrelin recognition by GOAT. The dansyl and acrylodan (AcDan) fluorophores used exhibit fluorescence enhancement upon a change in the polarity of the local environment, such as the increase in hydrophobicity expected upon octanoylation of serine 3 of ghrelin by hGOAT. Similar assays have proven robust for studying protein prenylation, another form of protein lipidation. The cysteine mutations at positions 2, 5, and 6 allow for AcDan conjugation at these sites, with mutations targeted to residues that are suggested to be non-essential for GOAT recognition. Fluorophore attachment was confirmed by HPLC and MALDI mass spectrometry, and milligram quantities of purified fluorescently labeled peptides may be produced.

With the peptide substrates, the addition of the hydrophobic octanoyl group upon serine acylation at position 3 leads to an increase in retention time in reverse-phase (RP) HPLC. The panel of fluorescent ghrelin peptide substrates was assayed for activity with hGOAT using HPLC coupled with fluorescence detection of the fluorescently labeled peptides. All reactions with hGOAT-His10 membranes were performed in parallel with mock membranes to account for nonspecific peptide modifications. Substrates with fluorophores attached N-terminal to the octanoylation site at serine 3 (1-Dansyl and AcDan-2) were not modified by hGOAT, whereas reactions with both the AcDan-5 and AcDan-6 substrates yielded the octanoylated peptide with increased retention time.

### EXAMPLE 1

An assay for human ghrelin O-acyltransferase activity was performed using hGOAT expressed from insect (Sf9) cells using standard baculoviral methods and a fluorescent 6-mer peptide synthesized by a single step reaction followed by high performance liquid chromatography (HPLC) purification. The assay was performed at room temperature using hGOAT membrane protein, octanoyl-CoA, a fluorescent ghrelin peptide, a buffer, and a detergent. Activity may be measure by detecting the presence of any octanoylation of the ghrelin peptide. For example, the presence of octanoylated ghrelin peptide may be confirmed by HPLC or by using a fluorescent system whose fluorescence will increase as a result of octanoylation of the ghrelin peptide.

### EXAMPLE 2

A library of potential inhibitors was screened using the assay of the present invention. More particularly, membrane fractions from Sf9 cells expressing hGOAT were thawed on ice and homogenized by passing through an 18-gauge needle ten times. Membrane fraction containing ˜50-100 μg membrane protein (determined by Bradford assay) was preincubated for 30 minutes at room temperature with 1 μM MAFP, 500 μM octanoyl CoA, 50 mM HEPES (pH=7.0), and 1 μl Diversity Set compound dissolved in DMSO for a final inhibitor concentration of either 10 or 100 μM for primary screening. Reactions were initiated with the addition of 1.5 μM fluorescent peptide substrate, incubated at room temperature in the dark for 3 hours, then stopped with 50 μl 20% acetic acid in isopropanol. The solutions were clarified by protein precipitation with 16.7 μl 20% trichloroacetic acid, followed by centrifugation (1,000×g, 1 m). The resulting supernatant was analyzed by reverse phase HPLC.

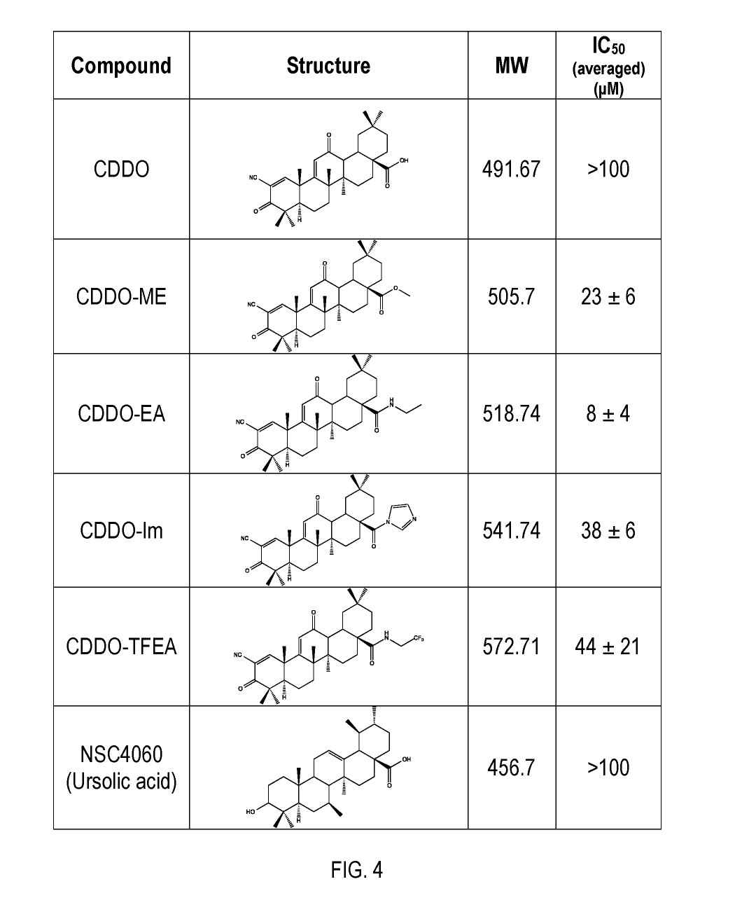

Once a compound was identified as demonstrating inhibitory activity against GOAT with an apparent IC₅₀<100 μM based on two trials of primary screen, a full inhibition titration was performed. Reactions followed the above procedure, and included inhibitor concentrations of 1, 5, 10, 50, or 100 μM, as well as a DMSO-only vehicle control. Compounds determined to act as hGOAT inhibitors are seen in FIGS. 4 through 6.

### EXAMPLE 3

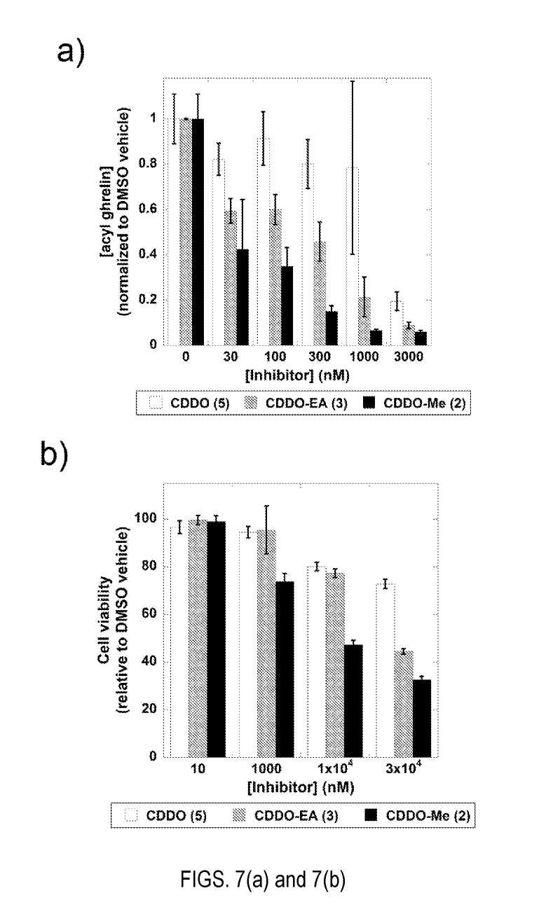

Referring to FIGS. 7 through 9, there are seen small molecule hGOAT inhibitors with demonstrated potency in both in vitro and cell-based assays. Bearing no resemblance to other known hGOAT inhibitors, these synthetic triterpenoids have the potential to be first-in-class therapeutics targeting ghrelin signaling. While several classes of small molecule inhibitors of GOAT have been previously reported in both the scientific and patent literature, the CDDO derivatives reported herein are the first examples of “drug-like” molecules with the validated ability to block ghrelin octanoylation within cells.

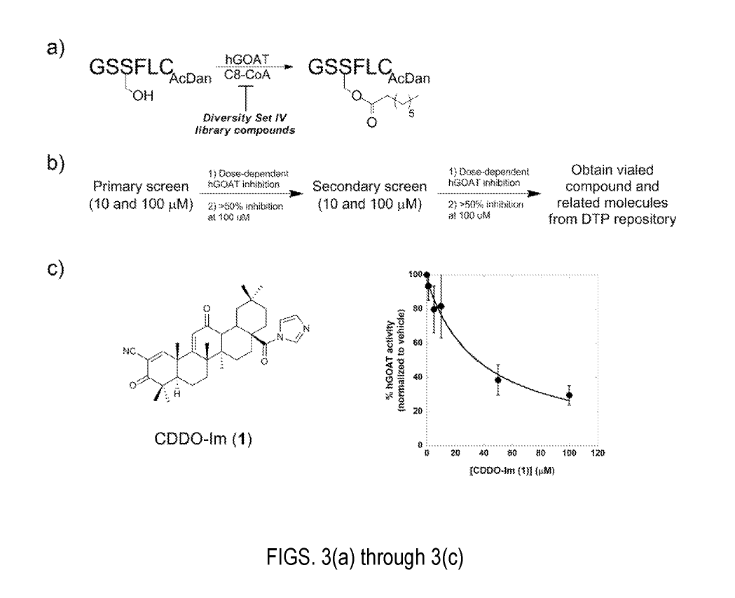

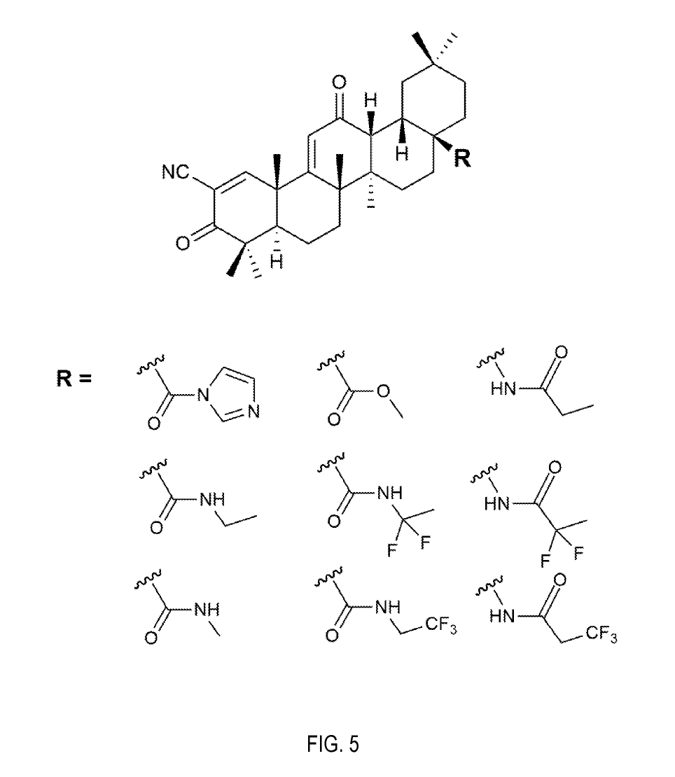

Following a screening protocol according to the present invention, the most promising candidate molecule was identified from the Diversity IV library as a synthetic oleanate triterpenoid, 1[2-cyano-3,12-dioxooleana-1,9-dien-28-oyl]imidazole (CDDO-Im, 1), as seen in FIG. 3. CDDO-Im inhibits hGOAT activity with an IC₅₀ of 38±6 μM and a structurally related molecule methyl 2-cyano-3,12-dioxooleana-1,9(11)dien-28-oate (CDDO-Me, 2) also exhibits inhibitory activity against hGOAT as seen in FIG. 4. As a result, structure of an inhibitor according to the present invention was determined, as seen in FIG. 5.

Verification of CDDO Scaffold Activity against hGOAT

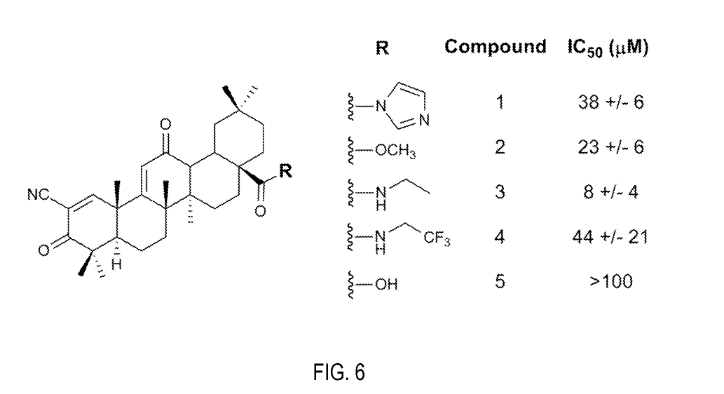

CDDO-Im and CDDO-Me belong to a class of orally available semisynthetic triterpenoids based on oleanolic acid. This class of compounds has demonstrated antiangiogenic and antitumor activities in animal cancer models through the modulation of multiple signaling pathways including the Nrf2 and NF-κB pathways. Given the inhibition of hGOAT exhibited by CDDO-Im and CDDO-Me, we determined the inhibitory activity of three other CDDO compounds with various carboxyl substituents (compounds 3-5, FIG. 6) against hGOAT using the in vitro hGOAT activity assay. Of these five CDDO compounds, all but the acid 5 served as inhibitors against hGOAT-catalyzed ghrelin octanoylation with the ethyl amide derivative (CDDO-EA, compound 3) demonstrating the most potent inhibition against hGOAT with an IC₅₀ of 8±4 μM (FIG. 6). The lack of inhibition activity exhibited by the parent CDDO molecule bearing a carboxylate may reflect a general intolerance for negatively charged groups within the hGOAT active site and substrate binding sites. Substrate selectivity studies of hGOAT have revealed hGOAT does not accept peptide substrates bearing negatively charged side chains or C-terminal acids.

Cell-Based Inhibition of Ghrelin Octanoylation by Synthetic Triterpenoids

Currently, no small molecule inhibitors have been reported to inhibit ghrelin acylation in vivo with only the peptide-based bisubstrate mimic GO-CoA-Tat shown to lower acyl ghrelin levels in cells and animal models. To evaluate the ability of these synthetic triterpenoids to block hGOAT-catalyzed ghrelin octanoylation in a biologically relevant context, we treated HEK293FT cells that have been stably transfected to express both preproghrelin and the mouse isoform of GOAT as seen in FIG. 7(a). Following treatment with CDDO (5), CDDO-EA (3), or CDDO-Me (2), cellular acyl ghrelin levels were determined by commercial ELISA analysis. While treatment with the parent acid CDDO did not markedly affect acyl ghrelin levels at concentrations up to 1 μM, both CDDO-EA (3) and CDDO-Me(2) effectively reduced acyl ghrelin concentrations with cellular IC₅₀ values of 320±150 nM and 35±10 nM, respectively as seen in FIG. 7(a). Inhibition of ghrelin acylation by CDDO derivatives occurs at compound concentrations below the onset of appreciable cytotoxicity with apparent LD₅₀ concentrations for both CDDO-EA (3) and CDDO-Me(2) lying near or above 10 μM for the stably transfected HEK293FT cell line utilized in this assay as seen in FIG. 7(b). The substantially lower IC₅₀ values observed for both CDDO-EA (3) and CDDO-Me(2) in the cell-based assay compared to the in vitro hGOAT activity assay could reflect less nonspecific compound sequestration into membrane structures or aggregates in cells compared to reactions involving high concentrations of microsomal protein fraction. As the cell-based assay more closely reflects the biologically relevant context for application of these molecules in a therapeutic setting, the submicromolar IC₅₀ values for CDDO-Me (2) and CDDO-EA (3) may indicate these molecules have sufficient cell permeability and potency against GOAT in a cellular context to support further investigation.

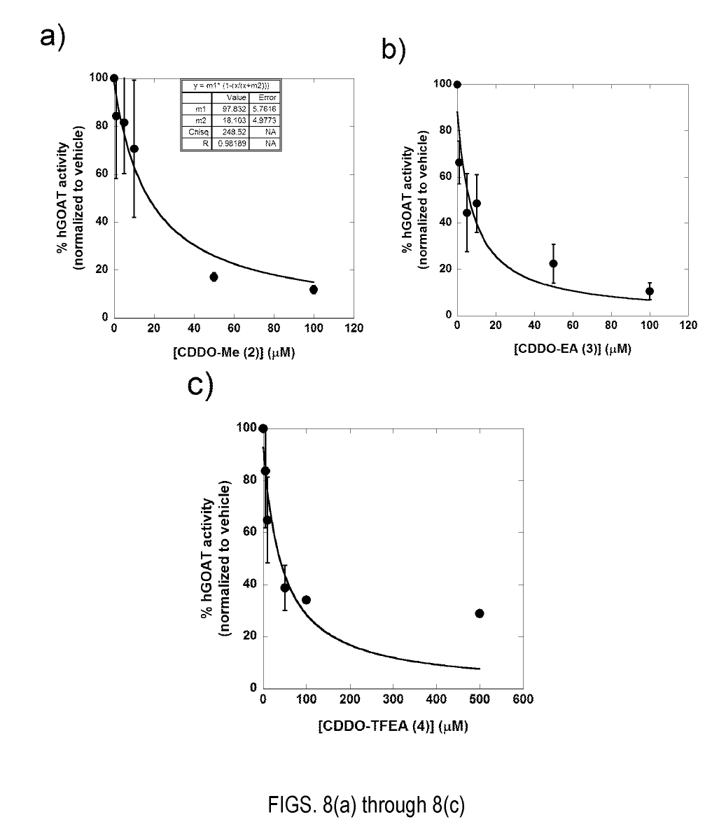

FIG. 8 shows the inhibition of hGOAT activity by the various CDDO derivatives, CDDO-Me (compound 2), CDDO-EA (compound 3), and CDDO-TFEA (compound 4) in a cell based assay. FIG. 8(c) show inhibition of hGOAT octanoylation activity by CDDO-FTEA (compound 4). These reactions were performed to determine percent activity with error bars reflecting the standard deviation from a minimum of three independent measurements.

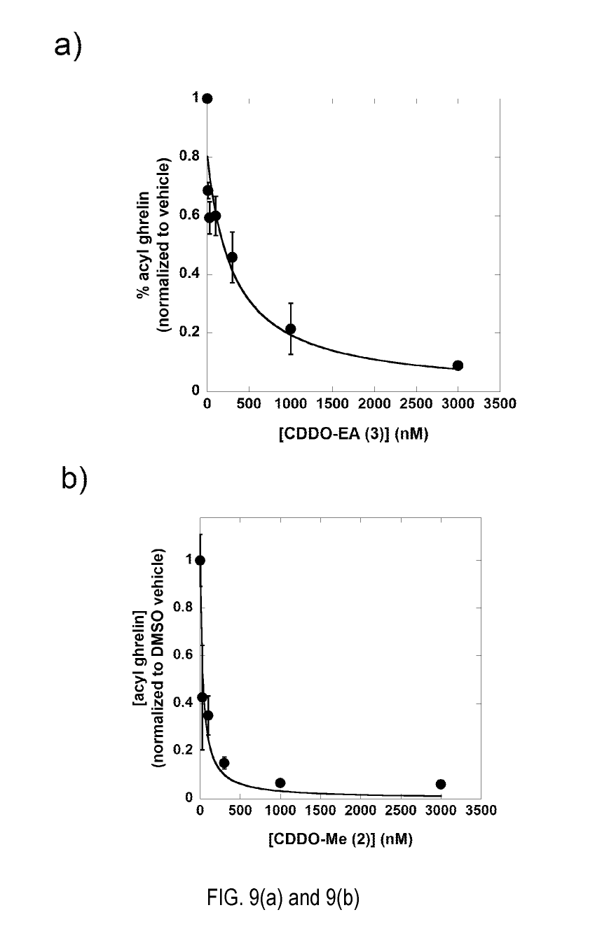

FIG. 9 shows the inhibition of ghrelin octanoylation by CDDO derivatives, CDDO-EA (compound 3) and CDDO-Me (compound 2) in a cell-based assay. Cell assays were performed to determine acyl ghrelin concentrations with error bars reflecting the standard deviation from a minimum of three independent measurements.

Discussion

Structure-activity analysis of the CDDO derivative inhibitors provides the first suggestion for the involvement of a functionally essential cysteine in GOAT catalyzed ghrelin acylation, and suggests these synthetic triterpenoids may function as the first reported mechanism-based inhibitors targeting GOAT. The regioselective requirement for an α,β-unsaturated ketone is consistent with inhibitor alkylation of a hGOAT cysteine residue acting as a Michael donor. Catalytic involvement of a cysteine in ghrelin acylation by hGOAT, while opening a new mode of inhibition targeting this enzyme, also presents an opportunity to potentially identify the location of the active site within an MBOAT-family acyltransferase. hGOAT contains a total of 16 cysteine residues, with several of these cysteines lying in the conserved C-terminal “MBOAT” domain within hGOAT. Mutational analyses of the three protein-modifying members of the MBOAT family (Hhat, PORCN, and GOAT) have revealed multiple functionally required residues but none have implicated cysteine residues as functionally essential. While Hhat and PORCN contain palmitoylated cysteine residues, these findings provide the first evidence supporting an enzymatic cysteine residue directly involved in MBOAT-catalyzed protein acylation. One intriguing possibility involves formation of an octanoyl acyl-enzyme intermediate involving a cysteine residue within GOAT in the course of transferring the octanoyl group to ghrelin, similar to the ping-pong mechanism proposed for protein palmitoylation by DHHC-family palmitoyltransfersases. Moving forward, studies to determine the identity and role(s) of functionally required cysteine residues within GOAT are currently underway.

The discovery of synthetic triterpenoid inhibitory activity against GOAT reveals an exciting and unanticipated mode of action for these compounds, several of which have been investigated in clinical trials. Previous studies of CDDO derivatives as potential therapeutics have focused on controlling inflammation and oxidative stress in multiple tissues through modulation of multiple cell signaling pathways. Inhibition of ghrelin acylation could explain multiple outcomes observed in rodent and human studies with these compounds, given ghrelin's known roles in regulating body energy balance and glucose metabolism. These outcomes observed during rodent and human studies utilizing CDDO derivatives, such as effects on fat deposition, weight loss, reduction of insulin resistance, and improved glucose tolerance, have been predicted as potential effects of modulating ghrelin signaling.

Identifying potent GOAT inhibitors is an essential step towards validation and exploitation of the ghrelin-GOAT system for therapeutic targeting. In this work, we demonstrate that synthetic triterpenoids containing an α-cyanoenone moiety can efficiently inhibit ghrelin acylation by GOAT in both in vitro and cell-based systems. These findings establish these CDDO derivatives as the first class of small molecule inhibitors for potential therapeutic targeting of ghrelin signaling and lay the foundation for future inhibitor development and optimization. As previous and ongoing clinical trials employing CDDO derivatives have established the suitability of these orally available compounds for human studies, we are hopeful that following animal studies to establish inhibitor efficacy in lowering acylated ghrelin serum concentrations these compounds can rapidly progress towards clinical trials as therapeutics for treating diabetes, obesity, and other health conditions impacted by ghrelin signaling.

Supplemental Information

General. Data plotting and curve fitting were performed with Kaleidagraph (Synergy Software, Reading, Pa., USA). Methoxy arachidonyl fluorophosphonate (MAFP) was purchased from Cayman Chemical (Ann Arbor, Mich.) as a stock in methyl acetate and diluted into DMSO prior to use. Octanoyl coenzyme A (octanoyl-CoA) was solubilized to 5 mM in 10 mM Tris—HCl (pH 7.0), aliquoted into low-adhesion microcentrifuge tubes, and stored at −80° C. Acrylodan (Anaspec) was solubilized in acetonitrile, with the stock concentration determined by absorbance at 393 nm on dilution into methanol (ε=18,483 M⁻¹ cm⁻¹ per manufacturer's data sheet). The GSSFLC_{NH2} peptide for fluorescent labeling with acrylodan were synthesized by Sigma—Genosys (The Woodlands, Tex., USA) in the Pepscreen format. The GSSFLC_{NH2} peptide was solubilized in 1:1 acetonitrile:H2O and stored at −80° C. Peptide concentration was determined spectrophotometrically at 412 nm by reaction of the cysteine thiol with 5,5′-dithiobis(2-nitrobenzoic acid) using ε₄₁₂=14,150 M-1 cm-1.

Library Screening

For screening from the Diversity Set IV library of small molecules, hGOAT octanoylation reactions were performed as described above with the addition of library compounds at concentrations of 10 and 100 μM, achieved by dilution of 10 mM compound stocks in DMSO received from the Developmental Therapeutics Program. Compounds that met criteria for inhibition (dose-dependent decrease in activity, <50% activity at 100 μM) were confirmed with a secondary screen using the same protocol.

Cell Line Generation and Culture

Stably transfected GOAT/Preproghrelin HEK 293FT cells were generated using the phPPG-mGOAT plasmid (a gift from Dr. Jef Boeke's laboratory) containing human preproghrelin (hPPG) and mouse ghrelin O-acyltransferase (mGOAT) connected by an intervening encephalomyocarditis virus internal ribosome entry site (ECMV-IRES) under puromycin selection as previously described. Cells were transfected using Lipofectamine 2000 (Invitrogen) at a 2 μL to 1 μg DNA ratio in 10 cm tissue culture-treated dishes according to manufacturer's protocol. The 293FT-hPPG-mGOAT cells were cultured in DMEM medium (Corning) supplemented with 10% inactivated fetal bovine serum, 1% penicillin/streptomycin, 1 ug/mL puromycin, and 0.01% octanoic acid and routinely passaged 2-3 times per week. A clonal line was chosen through serial dilution into a 96-well plate.

Inhibitor Treatment, Cell Lysate Preparation and Acyl Ghrelin Concentration Measurement by ELISA

Cell density and number were optimized for detection of acyl ghrelin via ELISA (EMD Millipore Corp). 293FT-hPPG-mGOAT clonal cells were plated in the late afternoon in 24-plates at 2×10⁵ cells/well. Cells were incubated overnight at 37° C., 5% CO₂ and treated the next morning with inhibitors or vehicle (DMSO) in triplicate. Cells were treated for 24 hours and harvested after medium aspiration using 40 μL/well trypsin-EDTA (0.25%, Corning) containing 5 μM methoxy arachidonyl flourophosphonate (MAFP). Cells were transferred to 1.5 mL siliconized tubes with 200 μL PBS containing 5 μM MAFP and centrifuged at 800×g for 5 min. The pellet was resuspended in 100 μL lysis buffer (50 mM Tris HCL pH 7.4, 150 mM NaCl, 2 mM EDTA, 1% NP-40, 0.1% SDS, Complete Protease Inhibitor Tab (Roche), 5 μM MAFP), vortexed for 3 seconds on high and frozen at −80° C. for a minimum of 16 hours. Immediately before use, lysates were thawed on ice, centrifuged at 20,000×g for 20 min at 4° C., and the supernatant was transferred to another 1.5 mL siliconized tube. Lysate was diluted 1:2 with lysis buffer before loading onto Human Active Ghrelin ELISA plates (EMD Millipore Corp). Samples were incubated with the capture and detection antibodies for 3.5 hours shaking at room temperature, with all other steps performed according to manufacturer's protocol.

Determination of Inhibitor Cytotoxicity by AlamarBlue Assay

293FT-hPPG-mGOAT cells were plated into 96-well plates at 2×10⁴ cells/well the evening before inhibitor addition. Cells were treated with inhibitor and 10% (v/v) AlamarBlue (Invitrogen) for 24 hours, followed by determination of cell viability according to the manufacturer's protocol using a Synergy H1 Hybrid plate reader (BioTek).

The compounds of the present invention may thus be used to treat various neurological processes that involve dysregulation of ghrelin signaling. For example, obesity, type II diabetes, gastroparesis, and regulation of appetite and body weight in patients with Prader-Willi syndrome are all known to involve dysregulation of ghrelin signaling and thus could be treated according to the present invention.

What is claimed is:

1. A method of inhibiting ghrelin O-acyltransferase, comprising the step of administering a synthetic oleanate triterpenoid.

2. The method of claim 1, wherein the synthetic oleanate triterpenoid comprises 2-cyano-3,12-dioxooleana-1,9(11)-dien-28-oic acid (CDDO).

3. The method of claim 2, wherein the synthetic oleanate triterpenoid comprises a derivative of 2-cyano-3,12-dioxooleana-1,9(11)-dien-28-oic acid (CDDO).

4. The method of claim 3, wherein the CDDO derivative comprises 1[2-Cyano-3,12-dioxooleana-1,9(11)-dien-28-oyl]imidazole (CDDO-Im).

5. The method of claim 3, wherein the CDDO derivative comprises methyl 2-cyano-3,12-dioxooleana-1,9(11)dien-28-oate (CDDO-ME).

6. The method of claim 3, wherein the CDDO derivative comprises 2-cyano-3,12-dioxooleana-1,9(11)-dien-28-oate-ethyl amide (CDDO-EA).

7. The method of claim 3, wherein the CDDO derivative comprises CDDO trifluoroethylamide (CDDO-TFEA).

8. A method of treating a patient having a disorder involving dysregulation of ghrelin signaling, comprising the step of administering a synthetic oleanate triterpenoid.

9. The method of claim 8, wherein the synthetic oleanate triterpenoid comprises 2-cyano-3,12-dioxooleana-1,9(11)-dien-28-oic acid (CDDO).

10. The method of claim 9, wherein the synthetic oleanate triterpenoid comprises a derivative of 2-cyano-3,12-dioxooleana-1,9(11)-dien-28-oic acid (CDDO).

11. The method of claim 10, wherein the CDDO derivative comprises 1[2-Cyano-3,12-dioxooleana-1,9(11)-dien-28-oyl]imidazole (CDDO-Im).

12. The method of claim 11, wherein the CDDO derivative comprises methyl 2-cyano-3,12-dioxooleana-1,9(11)dien-28-oate (CDDO-ME).

13. The method of claim 12, wherein the CDDO derivative comprises 2-cyano-3,12-dioxooleana-1,9(11)-dien-28-oate-ethyl amide (CDDO-EA).

14. The method of claim 13, wherein the CDDO derivative comprises CDDO trifluoroethylamide (CDDO-TFEA)

15. The method of claim 8, wherein the disorder is selected from the group consisting of obesity, weight gain, type II diabetes, gastroparesis, appetite dysregulation, anorexia nervosa, and symptoms related to elevated ghrelin levels in Prader-Willi syndrome.
